(19) United States
(12) Patent Application Publication
(10) Pub. No.: US 2007/0027544 A1
(43) Pub. Date: Feb. 1, 2007

(54) SPINAL CAGE IMPLANT

(75) Inventors: David McCord, Nashville, TN (US); John Kapitan, Waxhaw, NC (US); Jason Blain, Encinitas, CA (US)

Correspondence Address: CALFEE HALTER & GRISWOLD, LLP, 800 SUPERIOR AVENUE, SUITE 1400, CLEVELAND, OH, 44114, US

(73) Assignee: Altiva Corporation

(21) Appl. No.: 11/460,862

(22) Filed: Jul. 28, 2006

Related U.S. Application Data
(60) Provisional application No. 60/703,511, filed on Jul. 28, 2005.

(51) Int. Cl. A61F 2/44

(52) U.S. Cl. 623/17.11

(57) ABSTRACT

A spinal cage implant device includes a substantially planar top, a substantially planar bottom, two side walls, a posterior end wall, and an anterior end wall, wherein the implant is wider than high and longer than wide, and the substantially planar top and the substantially planar bottom include at least one opening, the external surfaces of the two side walls are textured and include at least one opening and that opening is bisected by at least one load-bearing frame structure, and at least one of the anterior end wall or posterior end wall include a hole for receiving a tool for surgical insertion of the spinal cage implant.

### PRIORITY CLAIM

This application claims priority to U.S. Provisional Patent Application No. 60/703,511 entitled “Spinal Cage Implant,” which was filed Jul. 28, 2005. Application No. 60/703,511 is incorporated herein by reference in its entirety.

### BACKGROUND OF THE INVENTION

The spinal column is formed from a number of vertebrae that are separated from one another by cartilaginous intervertebral discs. These discs form a cushion between adjacent vertebrae, resisting compression along the support axis of the spinal column, but permitting limited movement between the vertebrae to provide flexibility. Injury, disease, or other degenerative disorders may cause one or more intervertebral discs to deteriorate or become dislocated in some way. This damage can lead to compression of adjacent nerve roots, frequently causing chronic and often disabling pain.

A number of methods and associated devices have been suggested for the replacement of damaged intervertebral discs, and various methods of vertebral stabilization have been developed. For example, one common approach is to permanently stabilize or “fuse” adjacent vertebrae to maintain the proper intervertebral spacing and eliminate relative movement between the vertebrae. In this approach, a surgeon implants hollow plugs containing bone graft material between the vertebrae to encourage bone growth across the intervertebral space, with the objective of fusing the adjacent vertebra into one bone mass.

Vertebral stabilization of adjacent vertebrae utilizing fusion devices has proven successful in permanently preserving intervertebral spacing. However, design elements useful to permit bone growth into a fusion device are often sacrificed to satisfy the rigidity and support requirements of the device. And conversely, design elements useful to provide structural support between vertebrae are sacrificed to satisfy promotion of bone ingrowth. Thus, rigid spinal implant improvements are continually sought that effectively and permanently maintain intervertebral spacing to prevent nerve or spinal cord compression while allowing a maximal level of bone ingrowth into the interior of the device.

### SUMMARY OF THE INVENTION

The present invention provides spinal cage implants that are substantially rectangular in shape, having a top, a bottom, two side walls, and two end walls. A cage is a structure with an internal space and an openwork frame serving as support. In some embodiments, openings in the top and bottom and side walls with one or more load-bearing structural frames therein allow bone ingrowth through the interior chamber to facilitate fusion of the device and the adjacent vertebrae.

### BRIEF DESCRIPTION OF THE DRAWINGS

FIG. 1 is a side-elevational view of a lower portion of a human vertebral column shown in partial section to illustrate spinal cage implants according to one embodiment of the present invention.

FIG. 2 is a view of a portion of FIG. 1 viewed along the direction of line A-A of FIG. 1.

FIG. 3 is a transverse sectional view through line B-B of FIG. 2.

FIG. 4 is a anterior perspective view of one embodiment of the spinal cage implant according to this invention.

FIG. 5 is a posterior perspective view of the spinal cage implant of FIG. 4.

FIG. 6 is an anterior end view of the spinal cage implant of FIGS. 4 and 5.

FIG. 7 is a is a top plane view of the spinal cage implant of FIGS. 4 and 5.

FIG. 8 is a bottom plane view of the spinal cage implant of FIGS. 4 and 5.

FIG. 9 is a mid-line plane sectional view looking down from the top of one embodiment of the spinal cage implant trough section line C-C of FIG. 11.

FIG. 10 is a left-hand side view of the spinal cage implant of FIGS. 4 and 5.

FIG. 11 is a right-hand side view of the spinal cage implant of FIGS. 4 and 5.

### DETAILED DESCRIPTION OF THE INVENTION

The present invention will now be described with occasional reference to various embodiments of the invention. Unless otherwise defined, all terms used herein have the same meaning as commonly understood by one of ordinary skill in the art to that this invention belongs. The terminology used in the description of the invention herein is for describing particular embodiments only, and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing units of measure as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

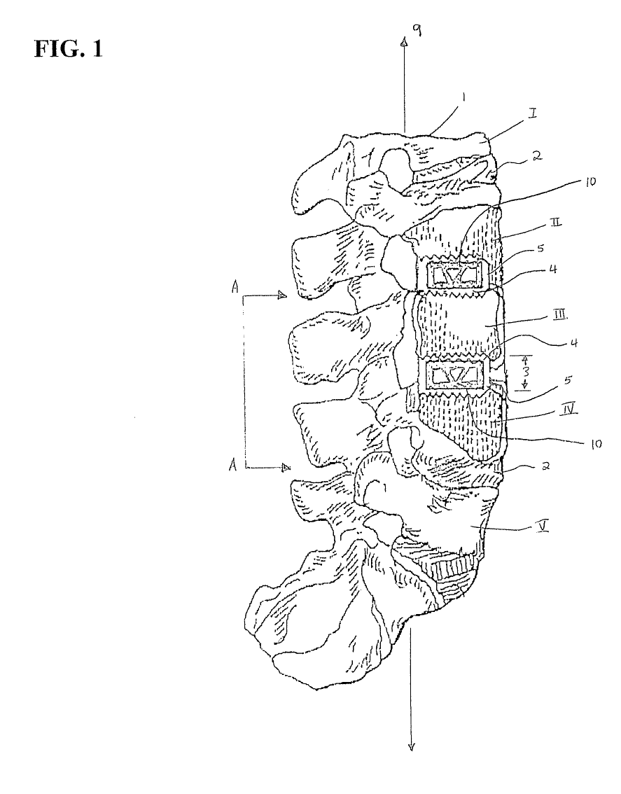

FIG. 1 illustrates one embodiment of the spinal cage implants 10 of the present invention, which are adapted to fit into disc spaces in portions of the spinal column. As depicted, the spinal cage implants 10 are inserted in the intervertebral spaces in the lower portion of the vertebral column 1, comprising the five lower vertebrae I, II, III, IV, and V. Adjacent vertebrae II and III and adjacent vertebrae III and IV are separated and supported by the spinal cage implants 10 of one embodiment of this invention.

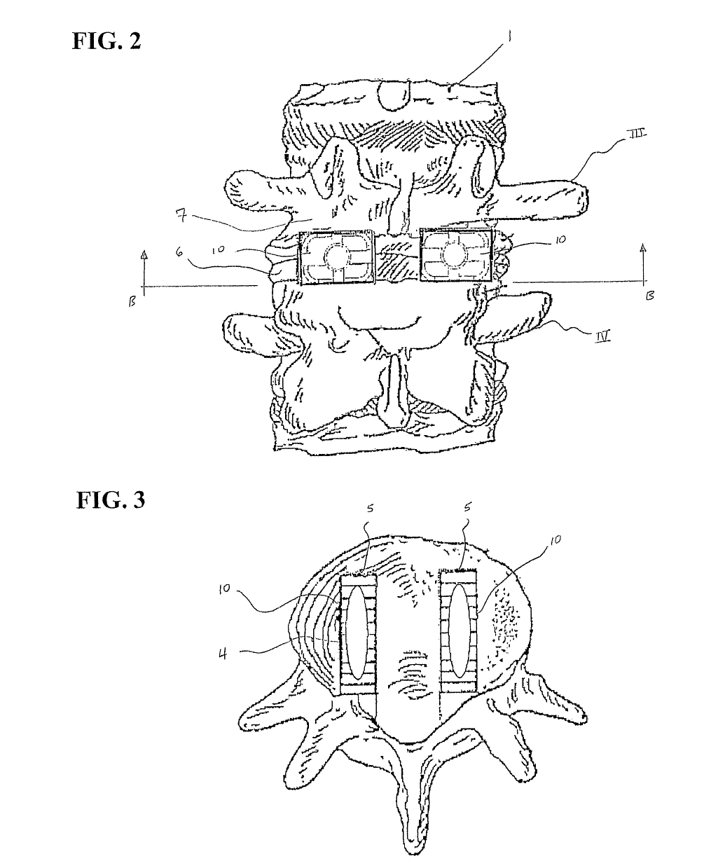

As further illustrated in FIG. 1 and FIG. 2, the spinal cage implants 10 replace portions of the natural human discs 2 between vertebrae II and III vertebrae III and IV. Disc spaces 3 are maintained by the prosthetic spinal cage implants 10 implanted therein. The opposed faces of adjoining vertebrae with damaged discs therebetween have aligned flat sided rectangular channels 4 cut therein transversely to axis 9 of the spinal column 1. The flat-sided, rectangular channels 4 have blind ends 5 that are abutted by the spinal cage implants 10. As shown in FIG. 2, the traverse channels 4 span the central soft cancellous bone 6, as well as the hard cortex bone 7 of the adjacent vertebrae. The human disc 2 remaining between the vertebrae may be trimmed to received the spinal cage implants 10.

As shown in the embodiments of FIG. 2 and FIG. 3, the spinal cage implants 10 are substantially rectangular, and are adapted to be inserted endwise into transverse channels 4 formed in the intervertebral spaces. As illustrated, more than one spinal cage implant 10 may be placed side by side within the intervertebral space. The spinal cage implant 10 size may vary based upon the size or anatomy of the patient requiring treatment. When implanted, the spinal cage implant 10 spans the soft cancellous bone 6 in the intervertebral space and rests on the hard cortex bone 7 of the adjacent vertebra.

Shape

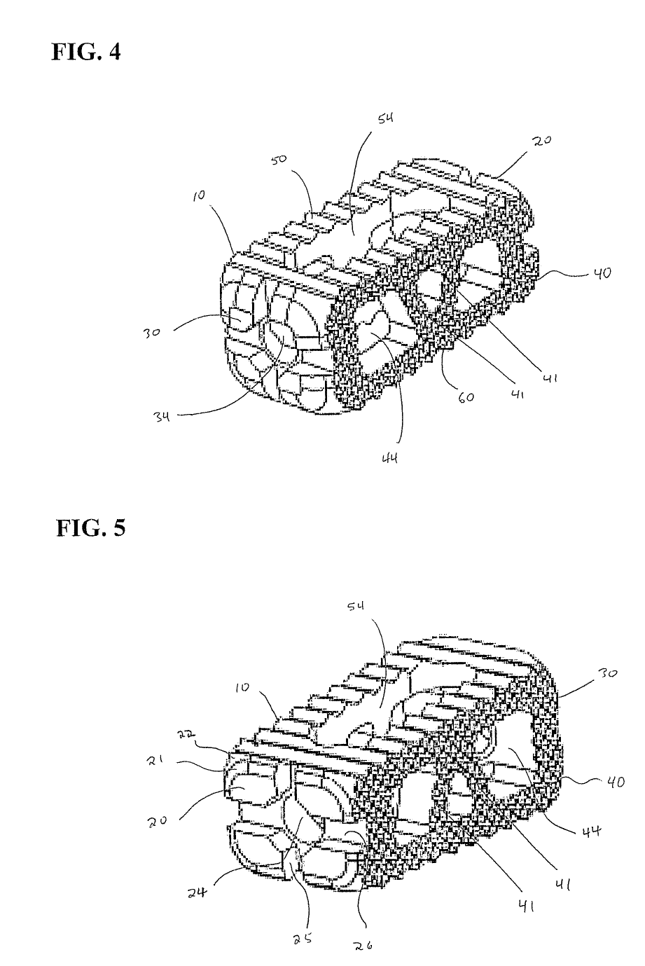

In the embodiment illustrated in FIGS. 4 and 5, the spinal cage implant 10 comprises a rigid cage that is substantially rectangular in shape, being wider than high and longer than wide. In various embodiments the spinal cage implant 10 has a substantially planar top 50, bottom 60, sides 40, anterior end wall 30, and posterior end wall 20. The anterior end wall 30 includes an anterior axial hole 34. The top 50 and bottom 60 of certain embodiments of the spinal cage implant 10 are perforated by substantially rectangular vertical openings 54. In the embodiment of FIGS. 4 and 5, the spinal cage implant 10 has horizontal openings 44 with unimpeded open ends in the textured sides 40 of the spinal cage implant 10. The horizontal opening 44 in this embodiment is rectangular and is bisected by a load-bearing structural frame 41.

In some embodiments, such as depicted in FIG. 4 and FIG. 5, the anterior end wall 30 is higher and wider than the posterior end wall 20. In other embodiments, the anterior end wall 30 and the posterior end wall 20 are the same size, and therefore the spinal cage implant 10 is parallelepiped. Further, in alternate embodiments, the anterior end wall 30 is higher but not wider or wider but not higher than the posterior end wall 20.

Sides

In certain embodiments of the present invention, the horizontal openings 44 in the sides 40 of the spinal cage implant 10 of may have rounded ends, semicircular ends, straight edges with right angles at the corners, or any combination thereof. The thickness of the sides 40 surrounding the horizontal opening 44 and the thickness of the load-bearing structural frame 41 are sufficient to maintain the height of the spinal cage implant 10 under full loads of the vertebrae under even the most adverse conditions. Embodiments of the invention may include at least one load-bearing structural frame 41 bisecting the horizontal opening 44.

In the illustrated embodiment of FIGS. 4 and 5, the load-bearing structural frame 41 comprises a truss within each horizontal opening 44 of the spinal cage implant 10. As seen in this particular embodiment, the load-bearing structural frame 41 on one side 40 may comprise the reverse orientation of the load-bearing structural frame 41 on the opposite side 40. In alternate embodiments not shown, the load-bearing structural frame 41 may comprise more than one truss on each side 40 of the spinal cage implant 10, and the trusses may have the same or reverse orientations.

In embodiments comprising more than one load-bearing structural frame 41, the frames 41 may be parallel or at an angle relative to each other, and the angle between them may be acute or obtuse. In alternate embodiments, the load-bearing structural frames 41 comprise the same configuration on both sides. In yet other embodiments, the load-bearing structural frame(s) 41 may be offset to one side, may comprise a different angle from the load-bearing structural frame(s) 41 on the opposite side, or may comprise any combination thereof. In certain embodiments, the load-bearing structural frame 41 may be textured similarly to the textured sides 20 of the spinal cage implant 40 as shown in FIGS. 4 and 5, whereas in other embodiments they may be smooth. In various embodiments, the left-side and right-side horizontal openings 44 may be similar or dissimilar, and variations may occur in any combination. In alternative embodiments, there could be two or more horizontal openings 44 so long as the overall area of the openings is sufficient to permit bone ingrowth.

Anterior and Posterior Ends

In the embodiment illustrated in FIG. 5, the posterior end wall 20 is beveled to a reduced substantially rectangular nose surrounded by flat sided tapered walls 21 with corners 22. The posterior end wall 20 is bisected by a top-to-bottom groove 25 and a perpendicular side-to-side groove 26, which promote bone ingrowth at the ends of the spinal cage implant 10. In certain embodiments, the grooves 25,26 further facilitate the coupling of the insertion tool and the spinal cage implant 10 during surgical insertion of the spinal cage implant 10. In the illustrated embodiment shown in FIG. 5, the posterior wall 20 also has an internally threaded posterior axial hole 24 at the center of the posterior end wall 20.

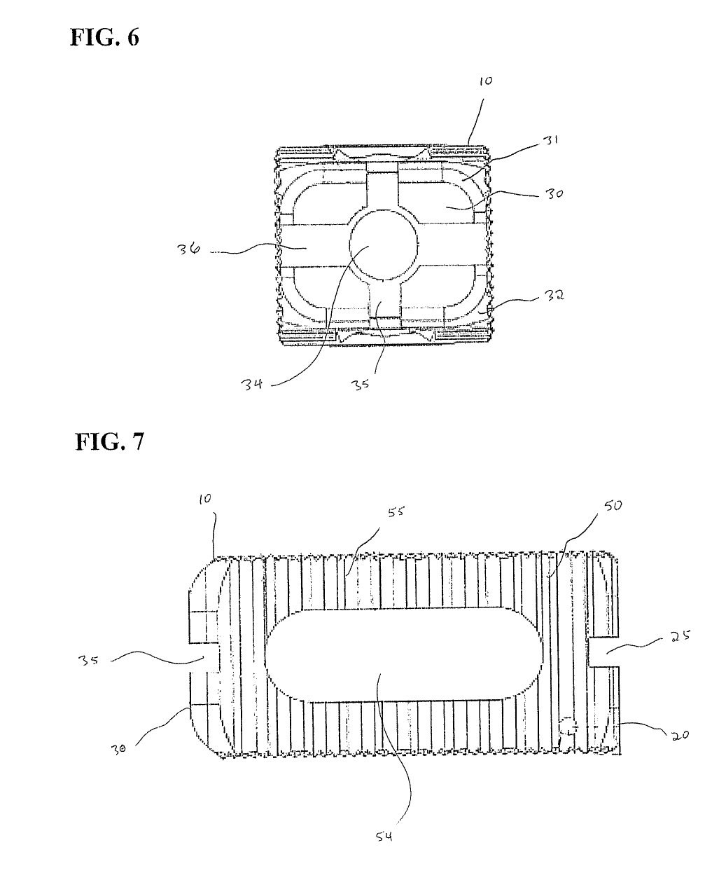

In the illustrated embodiment depicted in FIG. 6, the anterior end wall 30 is also beveled to a reduced substantially rectangular nose surrounded by flat sided tapered walls 31 with corners 32. In this embodiment, the anterior end wall 30 is bisected by a top-to-bottom groove 35 and a perpendicular side-to-side groove 36, which promote bone ingrowth at the ends of the spinal cage implant 10. In certain embodiments, the grooves 35,36 further facilitate the coupling of the insertion tool and the spinal cage implant 10 during surgical insertion of the spinal cage implant 10. In the illustrated embodiment, the anterior end wall 30 has an internally threaded anterior axial hole 34 at the center of the wall.

In alternate embodiments, the anterior end wall 30 may be substantially hemispherical or convex, and the nose may be convex or flat. The tapered portion of the anterior end wall 30 may extend to the edges of the anterior end wall 30 of the spinal cage implant 10 and the proportions of the tapered wall 31 and the corners 32 may vary inversely. In alternative embodiments, the anterior end top-to-bottom groove 35 or the anterior end perpendicular side-to-side groove 36 may be present alone, or both may be absent. In another embodiment, the anterior axial hole 34 may not be threaded. In yet another embodiments, the anterior end wall 30 may not be beveled.

In alternate embodiments, the posterior end wall 20 may be substantially hemispherical or convex, and the nose may be convex or flat. The tapered portion of the posterior end wall 20 may extend to the edges of the posterior end wall 20 of the spinal cage implant 10 and the proportions of the tapered wall 21 and the corners 22 may vary inversely. Alternatively, the posterior end top-to-bottom groove 25 or the posterior end perpendicular side-to-side groove 26 may be present alone or both may be absent. In another embodiment the posterior axial hole 24 may not be threaded. In yet another embodiment, the posterior end wall 20 may be not be beveled. In various embodiments, the anterior end wall 30 and the posterior end wall 20 may be similar or dissimilar, and variations may occur in any combination.

Side Texture

The side surfaces of certain embodiments of the spinal cage implants 10 are non-yielding and may have configurations to facilitate gripping transverse channels 4 and permitting bone ingrowth. In the embodiment illustrated in FIG. 4 and FIG. 5, side 40 surfaces of the spinal cage implants 10 are textured. In this particular embodiment, the texture is in the form of protrusions in the shape of four-sided pyramids. In alternate embodiments, the side 40 texture of the spinal cage implants 10 may comprise cross-hatching, pebbling, serrations, striations, or any combination thereof. In other alternate embodiments, the texture may comprise etching or recessed grooves, or the sides may be partially or entirely nontextured.

Top and Bottom

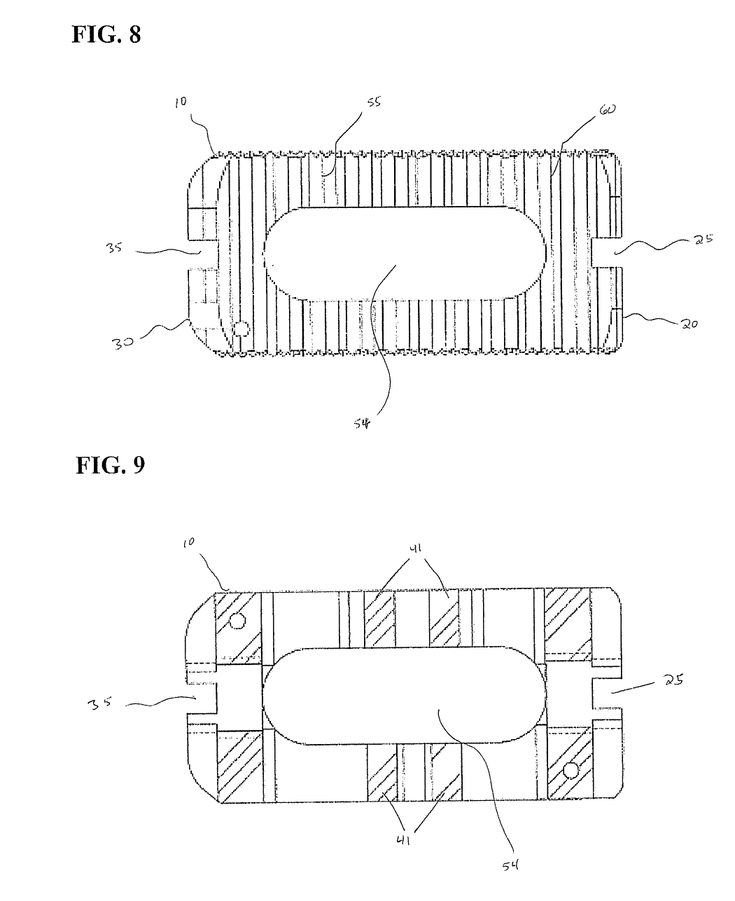

Depicted in FIG. 7 and FIG. 8, the top 50 and bottom 60 of the spinal cage implant 10 are perforated by vertical openings 54 that are substantially rectangular ovals. In other embodiments, the vertical openings 54 in the top 50 or bottom 60 may be substantially rectangular with sharp ends. In alternate embodiments, the vertical opening 54 may have rounded ends, semicircular ends, straight edges with right angles at the corners, or any combination thereof. In alternate embodiments, the vertical openings 54 may vary from one another, and those variations may occur in any combination.

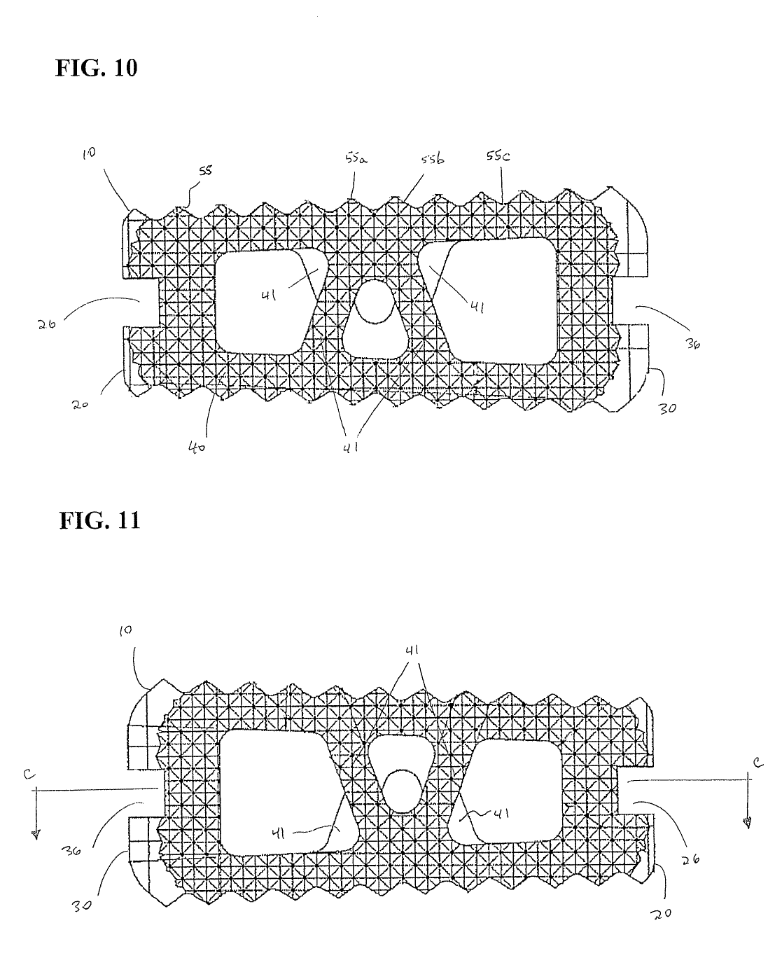

In the embodiment shown in FIGS. 7 and 8 the top 50 and bottom 60 have surface features 55 for interacting with the edges of the transverse channels. As shown in FIG. 10, this particular embodiment's surface features 55 are teeth or serrations, with the sides of the teeth 55b at obtuse angles from each other, and at acute angles to the top 50 or bottom 60. In this embodiment, the teeth have points 55a and spaces 55c between the points.

In certain embodiments the surface features 55 on the top 50 and the bottom 60 may be oriented towards the anterior end 30, the posterior end 20, straight up, or any combination thereof. In certain embodiments, the surface features 55 may extend across the full widths of the top 50 and bottom 60 of the spinal cage implant 10 to provide saw-like serrations. In yet other embodiments, the surface features 55 of the top 50 or bottom 60 may be in the form of nubs, ridges, spikes, pyramids, or other types of projections or protrusions.

In some embodiments, the heights of the posterior end wall 20 and anterior end wall 30 may be 7 mm, 8 mm, 9 mm, 10 mm, 11 mm, 12 mm, 13 mm, 14 mm, 15 mm, 16 mm, or 17 mm, any combination thereof. In certain other embodiments the height of the posterior end wall 20 may be 2 mm shorter than the height of the anterior end wall 30 with a continuous rise of 5° from end to end. In particular embodiments, the spinal cage implant 10 lengths will be 0.9 to 1.0 inches. In some embodiments, the length of the spinal cage implant 10 may be 0.905, 0.906, or 0.907 inches.

In one embodiment, surface features 55 on the top 50 or bottom 60 in the form of teeth projecting from the top or bottom surfaces will have heights of 0.01 to 0.02 inches with spaces 55c between the peaks 55a of 0.075 to 0.085 inches. In certain embodiments, the surface features 55 will have a height of 0.025 inches with a space 55c between the peaks 55a of 0.016 inches with an angle of 112° between the peaks 55a.

A typical horizontal opening 44 through the spinal cage implant 10 will extend substantially the full length of the spinal cage implant 10 and be wide or high enough to provide a length for the load-bearing structural frame truss of about 0.075 inches to 0.10 inches. In certain embodiments, the length is 0.081 inches. In other embodiments, the ends of the horizontal openings 44 are spaced inwardly from the leading and trailing ends of the opening to provide strong rigid truss support with widths from 0.08 to 0.2 inches. In certain embodiments, the end width is 0.157 inches.

In some embodiments, the anterior end wall 30 is beveled at an angle between 45° to 55°, including 45°, 46°, 47°, 48°, 49°, 50°, 51°, 52°, 53°, 54°, and 55° to a reduced substantially rectangular nose. In certain embodiments, the anterior end wall 30 angle is 50°. In certain other embodiments the flat sided tapered anterior end walls 30 are 0.094 inches wide and high. In some embodiments the anterior top-to-bottom groove 35 is approximately 0.062 inches wide and the perpendicular side-to-side groove 36 is approximately 0.082 inches wide. In certain embodiments, the anterior end hole 34 has a diameter of 0.164 inches.

In some embodiments, the posterior end wall 20 is beveled at angle between 40° to 45°, including 40°, 41°, 42°, 43°, 44°, and 45° to a reduced substantially rectangular nose. In certain embodiments, the posterior end wall 20 angle is 43°. In certain other embodiments the flat sided tapered posterior end walls 20 are 0.039 inches wide and high. In some embodiments the posterior top-to-bottom groove 25 is approximately 0.062 inches wide and the perpendicular side-to-side groove 26 is approximately 0.082 inches wide. In certain embodiments, the posterior end hole 24 has a diameter of 0.164 inches.

In some embodiments wherein the load-bearing structural frame 41 on the side 40 of the spinal cage implant 10 comprises a truss, the arms of the truss may have a width of 0.05 to 0.07 inches. In other embodiments, the angle between the arms of the truss may be between 25° to 55°, including 25°, 30°, 35°, 400, 45°, 50°, 55° or more. In several embodiments, the center of the truss located 0.4 to 0.5 inches from the posterior end wall 20. In certain embodiments, the arms of the truss have a width of 0.059 inches with a 40° angle between the arms. In certain other embodiments, the center of the truss is 0.433 inches from the posterior end wall 20.

In certain embodiments in which the side 40 texture is in the shape of four-sided pyramidal protrusions, the protrusions have a diameter of 0.01 to 0.05 inches and a height of 0.01 to 0.02 inches. In certain specific embodiments, the four-sided protrusions may have a diameter of 0.03 inches and a height of 0.015 inches with an angle of 90° between the peaks.

Material

In certain embodiments, the spinal cage implants may be made of a biologically compatible radiolucent material, such as a plastic of the nylon, polycarbonate, polypropylene, polyacetal, polyethylene, or polysulfone type, carbon fiber reinforced polymer such as PEEK (polyetherether ketone) or Ultrapek (polyether ketone ether ketone ketone), which may or may not be filled with glass or carbon fibers. These plastics can be injection molded, are lightweight, have great load carrying strength, and provide improved x-ray visualization of bone healing. In certain embodiments, the spinal cage implant is made of polycarbonate, polypropylene, polyethylene, or polysulfone types filed with glass or carbon fibers, such as supplied by ICI Industries of Wilmington, Del., Fiber-Rite Corporation of Winona, Minn., or BASF. Other embodiments may be composed of other biologically compatible orthopedic implant materials such as stainless steel, titanium, and chrome cobalt. One embodiment may be made of polyether sulfone filled with carbon fibers, such as supplied under the tradename “VICTREX P.E.S.,” including grade “4101 G.L,.-30” which is 30 percent fiber glass filled and “450 C.A.-30” which is 30 percent carbon fiber filled. These materials, are supplied from ICI Industries of Wilmington, Del. Also useful are the carbon-carbon fiber plastics of the type sold by Fiber-Rite Corporation of Winona, Minn.

In some embodiments, the interior of the spinal cage implant is packed with bone graft material prior to implantation. In yet other embodiments, the bone graft material is also packed between and beside the spinal cage implant and around the spinal cage implant in the disc space 3 between the vertebrae, or between and beside two or more adjacent spinal cage implants in full communication with their horizontal or lateral side openings which are also packed with bone graft material. The openings on the sides 40, top 50, and bottom 60 of the spinal cage implant 10, and the holes and grooves on the anterior and posterior end walls facilitate two-dimensional contact between bone graft material and disc tissue and expedite bone ingrowth and blood supply ingrowth from the sides to the bone between two spinal cage implants.

An insertion tool (not shown) is threaded into the anterior end hole 34 of the anterior end wall 30 of the spinal cage implant 10. The beveled or semicircular leading end of the spinal cage implant facilitates insertion of the spinal cage implant in proper position into the transverse channels 4 formed between the vertebrae. The insertion tool can thus gently guide the spinal cage implant into its position in the transverse channel 4. The insertion tool is then removed from the anterior end hole 34 of the spinal cage implant when the spinal cage implant is properly seated in the vertebrae channels or grooves.

The embodiments described above are examples of different embodiments and are not intended to limit the scope of the claims set forth below. Variations to the inventions described herein, including alternate embodiments not specifically described, are quiet possible and are encompassed by the claims as understood by one of ordinary skill in the art. Indeed, the claimed inventions have their broad and ordinary meaning as set forth below in the claims.

We claim:

1. A spinal implant for implantation within an intervertebral disc space comprising:
a substantially rectangular cage that is wider than high and longer than wide and having:
an interior space,
a substantially planar top and bottom, both comprising at least one opening,
two side walls, both comprising at least one opening, the at least one opening comprising at least one load-bearing structure,
a posterior end wall and an anterior end wall, at least one of which comprising a hole for receiving a tool for manipulation of the cage.

2. The spinal implant of claim 1, wherein the cage is composed of a rigid biocompatible material comprising a radiolucent material.

3. The spinal implant of claim 2, wherein the cage is composed of plastic or carbon fiber reinforced polymer.

4. The spinal implant of claim 1, wherein external surfaces of at least one of the substantially planar top and bottom is smooth.

5. The spinal implant of claim 1, wherein external surfaces of at least one of the substantially planar top and bottom is textured.

6. The spinal implant of claim 5, wherein the texturing comprises one or more of transverse ridges, grooves, and teeth.

7. The spinal implant of claim 1, wherein the anterior end wall is higher and wider than the posterior end wall.

8. The spinal implant of claim 1, wherein external surfaces of the side walls are smooth.

9. The spinal implant of claim 1, wherein external surfaces of the side walls are textured.

10. The spinal implant of claim 9, wherein the texturing comprises one or more of cross-hatching, pebbling, serrations, striations, and four-sided pyramid protrusions.

11. The spinal implant of claim 1, wherein the at least one load bearing structure includes at least one substantially triangular truss.

12. The spinal implant of claim 1, wherein the at least one load bearing structure includes two trusses.

13. The spinal implant of claim 12, wherein one truss is located within one opening in one side wall and the other truss is located within one opening in the other side wall, and the two trusses are similarly orientated to one another.

14. The spinal implant of claim 12, wherein one truss is located within one opening in one side wall and the other truss is located within one opening in the other side wall, and the two trusses are inversely orientated to one another.

15. The spinal implant of claim 1, wherein at least one of the anterior end wall or the posterior end wall further comprises at least one beveled protrusion forming a substantially rectangular nose, wherein the at least one beveled protrusion comprises at least one groove.

16. The spinal implant of claim 15, wherein the at least one beveled protrusion comprises two grooves that are oriented perpendicular to one another.

17. The spinal implant of claim 1, further comprising an osteogenic material within the interior space.

18. A spinal implant device for implantation within an intervertebral disc space comprising:
a substantially rectangular cage that is wider than high and longer than wide and having:
a substantially planar top and bottom, each comprising an opening, and each either smooth or textured,
two side walls, each of which comprises at least one opening, and wherein a truss is located within the at least one opening in one side wall and another truss is located within the at least one opening in the other side wall, and wherein the external surfaces of the two side walls are either smooth or textured, and
a posterior end wall and an anterior end wall, at least one of which comprising a hole for receiving a tool for surgical insertion of the spinal implant, and at least one of which comprising a beveled protrusion forming a substantially rectangular nose which includes at least one groove.

19. The spinal implant of claim 18, further comprising an interior space adapted for receiving an osteogenic material, and formed from a rigid biocompatible material that is radiolucent.

20. A spinal implant device for implantation within an intervertebral disc space comprising:
a substantially rectangular cage formed from a rigid biocompatible material that is radiolucent, and that is wider than high and longer than wide and having:
an interior space,
a substantially planar top and bottom, each comprising an opening, and each textured with transverse teeth,
two side walls, each of which comprises one opening, and wherein a truss is located within the one opening in one side wall and another truss is located within the opening in the other side wall, and the two trusses are inversely oriented to one another, and wherein the external surfaces of the two side walls are textured with four-sided pyramid protrusions,
a posterior end wall and an anterior end wall, each of which comprises a hole for receiving a tool for surgical insertion of the spinal implant, and each of which comprises a beveled protrusion forming a substantially rectangular nose which includes two grooves that are oriented perpendicular to one another, and
an osteogenic material within the interior space.
